(19) United States
(12) Patent Application Publication
(10) Pub. No.: US 2023/0338491 A1
(43) Pub. Date: Oct. 26, 2023

(54) METHOD FOR SCREENING INDIVIDUAL TUMOR NEOANTIGEN PEPTIDE, AND VACCINE FORMULATION THEREOF

(71) Applicant: HANGZHOU NEOANTIGEN THERAPEUTICS CO., LTD., Linping District (CN)

(72) Inventors: Fan MO, Binjiang District Hangzhou (CN); Shuiping SHI, Binjiang District Hangzhou (CN); Liang LIU, Binjiang District Hangzhou (CN); Min QIU, Binjiang District Hangzhou (CN); Ning HAN, Binjiang District Hangzhou (CN)

(21) Appl. No.: 18/338,605

(22) Filed: Jun. 21, 2023

Related U.S. Application Data
(63) Continuation of application No. PCT/CN2021/098629, filed on Jun. 7, 2021.

(30) Foreign Application Priority Data
Mar. 8, 2021 (CN) 202110251383.X

(51) Int. Cl. A61K 39/00; G16B 15/30; G16B 20/50; A61P 35/00; A61K 9/19; A61K 9/00; A61K 47/26; G16H 10/40

(52) U.S. Cl. CPC A61K 39/0011; G16B 15/30; G16B 20/50; A61P 35/00; A61K 9/19; A61K 9/0019; A61K 47/26; G16H 10/40

(57) ABSTRACT

A screening method of individualized tumor neoantigen peptide and a vaccine preparation thereof are provided. The screening method includes: Step 1, collecting and collating variable information for mutation producing a neoantigen and an antigenic peptide; Step 2, calculating according to a formula to obtain a score of each antigenic peptide; Step 3, arranging the antigen peptides in a descending order according to iNeo_Score, and selecting the antigen peptides from top to bottom successively; Step 4, continuing to select an antigenic peptide until enough candidate antigenic peptides are obtained or all of candidate antigenic peptides are selected so as to obtain screened antigenic peptides; and Step 5, grouping the screened antigen peptides into preparation groups. The designed individualized tumor neoantigen peptide is screened and prepared into a preparation in the disclosure, which includes screened antigen peptide, inorganic salt and an excipient. The preparation can be made into small-volume injection.

### TECHINCAL FIELD

The disclosure relates to the field of medicine, in particular to a screening method of an individualized tumor neoantigen peptide and a vaccine preparation thereof.

### BACKGROUND ART

As a kind of immunotherapy, tumor vaccine aims to aid an immune system in recognizing tumor cells and then to eliminate them. Tumor vaccines can be divided into preventive tumor vaccines and therapeutic tumor vaccines. The preventive tumor vaccines can not only aid bodies in training immune systems in advance without tumor infection in clinical applications, but also prevent recurrence of tumor after operations. The therapeutic tumor vaccines can activate immune response against existing tumor cells so as to eliminate lesions. The tumor vaccines can be divided into non-individualized vaccines and individualized vaccines according to sources of their antigens. The individualized vaccines utilize tumor-associated antigens (TAA) as an immune target to activate the immune response. The Tumor-associated antigens are common in both tumor cells and other normal human cells, which can be divided into 1) over-expression autoantigens (such as Her-2/neu, TERT and other antigens); 2) tissue differentiation antigens (such as PSA, Mammaglobin-A, Tryosinase and other antigens); 3) gonadal autoantigens (such as MAGE, Bag, NY-ESO-1 and other antigens) and 4) carcinoembryonic antigens (such as CEA, MUC-1, TPBG and other antigens). Many clinical trials aimed at traditional non-individualized tumor vaccines (vaccines developed based on the tumor-associated antigens) show that it is difficult for such anti-tumor vaccines to achieve long-term therapeutic effect. Neoantigens are usually produced by genome mutation of tumor cells and only exist in tumor cells, which are a kind of tumor specific antigen (TSA). Compared with the tumor-associated antigens, the neoantigens have high tumor specificity, usually stronger immunogenicity and better affinity for major histocompatibility complex (MHC protein), and are not affected by central immune tolerance, and thus having great potential in clinical treatment and application of tumors. Two research efforts of tumor vaccines based on neoantigens were published in Nature in 2017. Professor Catherine Wu's team from Dana-Farber Cancer Center in Boston, USA, and Ugur Sahin's team from Mainz University in Germany respectively demonstrated excellent efficacy of an individualized polypeptide vaccine and an individualized RNA vaccine based on tumor neoantigen in treatment of high-risk patients with advanced melanoma recurrence. These results provide strong evidence for safety, immunogenicity and effectiveness of individualized tumor neoantigen vaccines.

The tumor vaccines can be divided into dendritic cell (DC) vaccines, nucleic acid (DNA or RNA) vaccines, protein vaccines and polypeptide vaccines. The polypeptide vaccines have advantages of easy synthesis and purification, safe application and no potential carcinogenicity, and many kinds of polypeptide vaccines have been marketed at home and abroad. At present, there are also several ongoing clinical trials related to the tumor neoantigen polypeptide vaccines. The key to success of polypeptide vaccine therapy for the tumor neoantigens lies in: 1) separating neoantigens that can specifically stimulate immune cells from sequencing data of tumor patients; 2) designing presentable epitope sequences contained in these neoantigens into vaccine polypeptide sequences which are easy to synthesize; 3) transport and store the polypeptide vaccines reasonably, and finally input them into patients to make it effective. A polypeptide is a compound formed by amino acids linked together by peptide bonds. A sequence of amino acid residues in a polypeptide chain is a most basic structure of the polypeptide, which determines a secondary and tertiary structure and various physical and chemical properties of the polypeptide. As for tumor neoantigen polypeptides, due to their special properties of individualized customization, a basic structure of each polypeptide, including an amino acid sequence of a neoantigen epitope sequence, is different, which determines that the designed and prepared tumor neoantigen polypeptides often have different physical and chemical characteristics, such as isoelectric point, hydrophilicity and hydrophobicity, solubility, redox, pH value, peptide chain stability and so on. All these bring great difficulties to preparation, storage, transportation and clinical use of the tumor neoantigen polypeptide vaccines. In order to ensure qualified quality and long shelf life of the individualized tumor neoantigen polypeptide vaccines, realize stable storage and ensure vaccine efficacy, while avoiding trouble of developing different processes for different antigenic peptides, a set of universal and feasible preparation processes is needed to be developed to prepare the tumor neoantigen polypeptide vaccines. Administration routes of polypeptide tumor vaccines include subcutaneous injection, intravenous injection and intramuscular injection. The subcutaneous injection is a first choice for tumor vaccine administration at present. Therefore, a general and feasible preparation process for the tumor neoantigen polypeptide vaccines is needed to be developed. The present disclosure solves such problems.

### SUMMARY

In order to solve shortcomings of related art, the disclosure aims to provide a screening method of an individualized tumor neoantigen peptide and a vaccine preparation thereof. The designed individualized tumor neoantigen peptide is screened and prepared into a preparation in this disclosure, which has excellent tumor inhibition effect.

In order to achieve above objects, the disclosure employs the following technical scheme.

A screening method of an individualized tumor neoantigen peptide includes following steps:
step 1, collecting and collating variable information for mutation producing a neoantigen contained in a vaccine and an antigenic peptide;
the variable information including: a mutation frequency Ag of the mutation producing the neoantigen at a genome level, a mutation frequency Ar of the mutation producing the neoantigen at a transcriptome level, expression level E of a gene where the mutation producing the neoantigen is located, a number H of amino acid changes caused by the mutation, quality indexes Mᵢ and Mᵢᵢ of epitope sequences of MHC protein type I and MHC protein type II, a situation ACTIVE where the antigen peptide contains active peptide, a situation DRUG where the antigen peptide contains drug peptide, homology HOM of the antigen peptide with normal human protein, and toxicity prediction TOXIC of the antigen peptide;
step 2, calculating according to a formula to obtain a comprehensive score iNeo_Score of each designed antigenic peptide;
the comprehensive score iNeo_Score being calculated as follows:

iNeo_Score=f₁(Ag)×f₂(Ar)×f₃(E)×f₄(Mᵢ)×f₅(H)+f₆(Mᵢᵢ);
where Ag represents the mutation frequency of the mutation producing the neoantigen at the genome level, Ar represents the mutation frequency of the mutation producing the neoantigen at the transcriptome level, E represents the expression level of the gene where the mutation producing the neoantigen is located, H represents the amino acid change caused by the mutation, Mᵢ and Mᵢᵢ represent type I and type II quality indexes of epitope sequences calculated by combining the epitope sequences of MHC protein type I and MHC protein type II, and f1 to f6 represent conversion functions of respective indexes;
removing an antigenic peptide with iNeo_Score of 0, and taking all of remaining antigenic peptides as candidate antigenic peptides for antigenic peptide screening;
step 3, arranging the antigen peptides in a descending order according to iNeo_Score, and then selecting the antigen peptides from top to bottom successively; and examining three indexes, namely, the toxicity, the active peptide and the homology according to peptide segment information after selecting an antigenic peptide;
directly discarding the antigenic peptide if any of above three indexes of the antigenic peptide is unqualified; and
reserving the antigen peptide as a selected antigen peptide and deleting other antigen peptide sequences generated by a same mutation if all of the above three indexes of the antigenic peptide are qualified;
step 4, continuing to select a next antigenic peptide downward in the descending order, and repeating above steps until enough candidate antigenic peptides are obtained or all of candidate antigenic peptides are selected so as to obtain screened antigenic peptides;
step 5, synthesizing a polypeptide, collecting a part with purity greater than threshold purity in segments with a purification system, which is concentrated and freeze-dried after a sufficient amount is collected, placing freeze-dried products for purity detection after drying, and if purity of this peptide is still higher than the threshold purity, selecting the peptide into a preparation group, and if the purity of the peptide is lower than the threshold purity, determining the peptide as an unstable peptide and taking the peptide into the preparation group; and
step 6, grouping the screened antigenic peptides into the preparation group;
grouping requirements including that:
1. n polypeptides are grouped and a number of groups is determined according to a number of the polypeptides;
2. peptides divided into each group are ranked according to corresponding HPLC retention time in an ascending order according to HPLC retention time corresponding to each peptide, and difference of retention time between two adjacent polypeptides in each group is greater than time difference corresponding to a maximum peak width of a single polypeptide peak;
3. polypeptides with cysteine are evenly divided into each group; and
4. each peptide has a corresponding gel chromatography polymer retention time, and it is required that gel chromatography polymer retention time corresponding to all of raw peptides divided into each group does not overlap with gel chromatography retention time of all of the raw peptides themselves.

In the screening method of the individualized tumor neoantigen peptide described above, information sources of variables include:
information source of the mutation frequency Ag of the mutation producing the neoantigen at the genome level being exon sequencing,
information source of the mutation frequency Ar of the mutation producing the neoantigen at the transcriptome level being transcriptome sequencing,
information source of the expression level E of the gene where the mutation producing the neoantigen is located being transcriptome sequencing,
information source of a number H of amino acid changes caused by the mutation being mutation information annotation,
the quality indexes Mᵢ and Mᵢᵢ of the epitope sequences of the MHC protein type I and MHC protein type II comprehensively considering a number of the epitope sequences, affinity of the epitope sequences with MHC protein, and affinity change of the epitope sequences with MHC protein; and their information source being the affinity prediction of the epitope sequences with MHC protein,
information source of the situation ACTIVE where the antigen peptide contains active peptide being antigenic peptide information annotation,
information source of the situation DRUG where the antigen peptide contains drug peptide being the antigenic peptide information annotation,
information source of homology HOM of the antigen peptide with normal human protein being the antigenic peptide information annotation, and
information source of toxicity prediction TOXIC of the antigen peptide being toxicity prediction analysis of the antigenic peptide.

In the screening method of the individualized tumor neoantigen peptide described above, three indexes, namely, the toxicity, the active peptide and the homology, in the step 3 are respectively as follows:
an active peptide index: amino acid sequences of the antigen peptide contains an active peptide amino acid sequence or a drug peptide amino acid sequence, and the active peptide amino acid sequence or the drug peptide amino acid sequence contains amino acid sites for mutation;
a toxicity index: the toxicity prediction of the antigen peptide is toxic; and
a homology index: homology of the antigenic peptide with human protein other than the gene where the mutation is located is over 80%.

In the screening method for the individualized tumor neoantigen peptide described above, specific rules for n polypeptides being grouped and a number of groups being determined according to a number of the polypeptides in the step 6 are as follows:
a number of antigenic peptides is set to be n,
if the number of the antigenic peptides is n>20, the number of groups is a value of n divided by 5 and then rounded up;
if the number of the antigenic peptides is 16<=n<=20, the number of groups is 4;
if the number of the antigenic peptides is 11<=n<=15, the number of groups is 3; and
if the number of the antigenic peptides is 5<=n<=10, the number of groups is 2.

In the screening method for the individualized tumor neoantigen peptide described above, specific rules for grouping the screened antigenic peptides into the preparation group in step 6 are as follows:
in a first step,
the number of the antigen peptides is known to be p, and according to grouping rules, the number of the groups is determined to be g, and the number of the antigen peptides in each group is expressed by aᵢ, i being 1, 2, 3, . . . , g; a₁+a₂+ . . . +a_{g}=p. The computer system can list all possible grouping results (a₁, a₂, . . . , a_{g}) of polypeptides divided into each group according to two data of p and g, the system can calculate variance of each group according to grouping results of the polypeptides divided into each group, and rank all of the grouping results of the polypeptides according to the variance in a descending order. The more average the grouping results are, the smaller the variance is, and a group with a most average grouping result has a smallest variance and can be ranked first. Then, according to a variance ranking order, the system sequentially perform full permutation C_{P}^{a}^{₁} C_{P-a}_{₁}^{a}^{₂} C_{P-a}_{₁}₋ₐ_{₂}^{a}^{₃} . . . C_{P-a}_{₁}₋ₐ_{₂}₋ₐ_{₃} _{. . . -a}_{_{g-1}}^{a}^{_{g}} on the grouping results of the polypeptides divided into each group while checking conditions in second, third and fourth steps. When calculating the grouping results of the polypeptides divided into each group, the system quickly finds largest HPLC retention time of an antigen peptide that can be successfully grouped with dichotomy and groups the antigen peptide, and if the antigen peptide cannot be found, a next grouping result of polypeptides is calculated until grouping is successful;
in a second step, it is checked whether polypeptides with cysteine are evenly distributed in each group, and if the polypeptides with cysteine are not evenly distributed in each group, grouping is continued, if the polypeptides with cysteine are evenly distributed in each group;
in a third step, it is checked whether the gel chromatography polymer retention time corresponding to each peptide overlaps with retention time of a main peptide, and if the gel chromatography polymer retention time corresponding to each peptide does not overlap with the retention time of the main peptide, requirements are met;
if the retention time of the gel chromatography polymer corresponding to each peptide overlaps with retention time of the main peptide, grouping is continued until a successful grouping is obtained; and
in a fourth step, according to a formula of vaccine preparation, solubility of the preparation group in the third step is rechecked, and if the preparation group is soluble, the grouping is successful, and if the preparation group is insoluble, a grouping method in the third step is repeated for a second time until the solubility rechecking result indicates soluble so as to obtain successful grouping.

A vaccine preparation of the individualized tumor neoantigen peptide described above includes, in parts by mass, 1-3 parts of screened antigen peptide, 0-20 parts of inorganic salt and 10-100 parts of an excipient.

In the vaccine preparation of the individualized tumor neoantigen peptide described above, the excipient includes: a cosolvent, a filler or a tonicity adjusting agent.

In the vaccine preparation of the individualized tumor neoantigen peptide described above, the cosolvent includes: saccharide or polyol adjuvant.

In the vaccine preparation of the individualized tumor neoantigen peptide described above, the cosolvent is mannitol.

In the vaccine preparation of the individualized tumor neoantigen peptide described above, the vaccine preparation is a small-volume injection; a concentration of each antigenic peptide is 0.1-0.5 mg/ml, and a concentration of the cosolvent is 0.5%-5% (w/v).

In the vaccine preparation of the individualized tumor neoantigen peptide described above, the vaccine preparation is freeze-dried powder injection; and a freeze-drying method includes following steps:
a, placing a bottle filled with vaccine liquid medicine into a freeze-drying dryer;
b, starting the freeze-drying dryer, and lowering a temperature of heat transfer oil of the freeze-drying dryer;
c, starting vacuumizing;
d, continuing to heat up a partition when vacuum reaches a specified requirement, e, continuously adjusting the temperature of the heat transfer oil to heat up;
f, continuing to heat up the partition when vacuum reaches a specified requirement, and
g, closing the freeze-drying dryer, charging nitrogen, plugging and discharging.

The disclosure has advantages as follows.

The designed individualized tumor neoantigen peptide is screened and prepared into a preparation in the disclosure, which has excellent tumor inhibition effect.

In the disclosure, by improving formulation of the preparation, small-volume injection and freeze-dried powder are included in a general aseptic preparation method of individualized tumor vaccine;

In the disclosure, the mannitol can be used not only as a filler of freeze-dried preparations, but also as the cosolvent of insoluble polypeptides, thus greatly improving solubility of hydrophobic peptides in aqueous solvents.

### BRIEF DESCRIPTION OF THE DRAWINGS

FIG. 1 shows treatment results of a C57-B16F10 melanoma model in experiments of the disclosure (A: a tumor growth curve of the melanoma treatment model; B: a total life cycle of the melanoma treatment model);

FIG. 2 shows treatment results of a Balb/c-CT26.wt colon cancer model in experiments of the disclosure (A: a tumor growth curve of the colon cancer treatment model; B: a total life cycle of the colon cancer treatment model);

FIG. 3 shows respective proportions of IFN-γ+ cells in a treatment model in experiments of the present disclosure; and

FIG. 4 is a flowchart of an embodiment of a screening method according to the present disclosure.

### DETAILED DESCRIPTION

The disclosure will be described in the following in detail with reference to the drawings and specific embodiments.

As shown in FIG. 4, a screening method of an individualized tumor neoantigen peptide includes following steps 1 to 6.

In step 1, variable information is collected and collated for mutation producing a neoantigen contained in a vaccine and an antigenic peptide.

TABLE 1
List of variables required for antigenic peptide scoring

| Variable | | |
|---|---|---|
| name | Variable meaning | Information source |
| Ag | Mutation frequency Ag of | Exon sequencing |
| | mutation producing the | |
| Ar | Mutation frequency of the | transcriptome sequencing, |
| | mutation producing the | |
| E | Expression level E of the gene | transcriptome sequencing, |
| | where the mutation producing | |
| H | Number of amino acid changes | Mutation information |
| | caused by the mutation | annotation |
| Mᵢ | Quality index of epitope | Afinity prediction of |
| | sequences of MHC protein type | epitope sequences |
| Mᵢᵢ | Quality index of epitope | Afinity prediction of |
| | sequences of MHC protein type | epitope sequences |
| ACTIVE | Situation where the antigen | Antigenic peptide |
| | peptide contains active peptide | information annotation |
| DRUG | Situation where the antigen | Antigenic peptide |
| | peptide contains drug peptide | information annotation |
| HOM | Homology of the antigen peptide | Antigenic peptide |
| | with normal human protein | information annotation |
| TOXIC | Toxicity prediction of the | Toxicity prediction analysis |
| | antigen peptide | of the antigenic peptide |

Remarks: Presentation indicates that the epitope sequences and the WIC protein form a pMHC protein complex, which is then presented as a whole to immune cells for recognition, so it is necessary to predict the affinity of the epitope sequences with the WIC protein.

In step 2, calculation is performed according to a formula to obtain a comprehensive score iNeo_Score of each designed antigenic peptide. An antigenic peptide with iNeo_Score of 0 is removed, and all of remaining antigenic peptides are taken as candidate antigenic peptides for antigenic peptide screening.

The comprehensive score iNeo_Score is calculated as follows:

iNeo_Score=f₁(Ag)×f₂(Ar)×f₃(E)×f₄(Mᵢ)×f₅(H)+f₆(Mᵢᵢ);
where Ag represents the mutation frequency of the mutation producing the neoantigen at the genome level, Ar represents the mutation frequency of the mutation producing the neoantigen at the transcriptome level, E represents the expression level of the gene where the mutation producing the neoantigen is located, H represents the amino acid change caused by the mutation, Mᵢ and Mᵢᵢ represent type I and type II quality indexes of epitope sequences calculated by combining the epitope sequences of MHC protein type I and MHC protein type II, and f1 to f6 represent conversion functions of respective indexes.

In step 3, the antigen peptides are arranged in a descending order according to iNeo_Score, and then the antigen peptides are selected from top to bottom successively; and following three indexes, namely, the toxicity, the active peptide and the homology are examined according to peptide segment information after an antigenic peptide is selected.

An active peptide index: amino acid sequences of the antigen peptide contains an active peptide amino acid sequence or a drug peptide amino acid sequence, and the active peptide amino acid sequence or the drug peptide amino acid sequence contains amino acid sites for mutation;

A toxicity index: the toxicity prediction of the antigen peptide is toxic; and

A homology index: homology of the antigenic peptide with human protein other than the gene where the mutation is located is over 80%. It should be noted that the antigenic peptide is formed after mutation of EGFR gene, because the mutation is generally a point mutation with only one amino acid change, similarity between this new antigenic peptide and a wild-type EGFR is very high, so the antigenic peptide needs to have no high homology with human gene protein sequences other than EGFR gene.

If any of above three indexes of the antigenic peptide is satisfied, and any of the above three indexes of the antigenic peptide is unqualified, the antigenic peptide is directly discarded.

If all of the above three indexes of the antigenic peptide are not satisfied, all of the above three indexes of the antigenic peptide are qualified and the antigen peptide is reserved as a selected antigen peptide and other antigen peptide sequences generated by a same mutation is deleted (which can be excluded according to mutation numbers).

A screening process of neoantigen peptide is actually shown in the following.

There are 10 candidate neoantigenic peptides in Table 2 below, and the table also contains mutation and antigenic peptide related information necessary for antigenic peptide screening.

Firstly, the 10 antigenic peptides were arranged in a descending order according to iNeo_Score, and then selected successively from top to bottom.

TABLE 2

| PID | VacSeq | iNeo_Score | VacRank | MutRank | Active | Drug | Hom | Toxic |
|---|---|---|---|---|---|---|---|---|
| P0001017 | GYLREEMV | 1.000 | 1 | 1 | — | 45.5% | 90.9% | No toxic |
| | LDIIPKLLNC | | | | | | | (non- |
| | LRDK | | | | | | | toxic) |
| P0001016 | GYLREEMV | 1.000 | 2 | 1 | — | 45.5% | 90.9% | No toxic |
| | LDIIPKLLNC | | | | | | | (non- |
| | LRDK | | | | | | | toxic) |
| P0000578 | KKLVFLLPA | 0.293 | 3 | 2 | — | 27.6% | 51.7% | No toxic |
| | ALFPRKKLFI | | | | | | | (non- |
| | TAAIVKKKK | | | | | | | toxic) |
| | K | | | | | | | |
| P0000774 | NSDFNFDNV | 0.137 | 4 | 3 | — | 30% | 86.7% | No toxic |
| | LSAMMTLFT | | | | | | | (non- |
| | VSTFEGWPA | | | | | | | toxic) |
| | KKK | | | | | | | |
| P0000772 | NSDFNFDNV | 0.137 | 5 | 3 | — | 30% | 86.7% | No toxic |
| | LSAMMTLFT | | | | | | | (non- |
| | VSTFEGWPA | | | | | | | toxic) |
| | KKK | | | | | | | |
| P0000771 | NSDFNFDNV | 0.137 | 6 | 3 | — | 30% | 86.7% | No toxic |
| | LSAMMTLFT | | | | | | | (non- |
| | VSTFEGWPA | | | | | | | toxic) |
| | KKK | | | | | | | |
| P0000773 | NSDFNFDNV | 0.137 | 7 | 3 | — | 30% | 86.7% | No toxic |
| | LSAMMTLFT | | | | | | | (non- |
| | VSTFEGWPA | | | | | | | toxic) |
| | KKK | | | | | | | |
| P0000526 | PGSALELRY | 0.092 | 14 | 4 | — | 53.3% | 43.8% | No toxic |
| | SQAPTVPSG | | | | | | | (non- |
| | AHLDPYVAG | | | | | | | toxic) |
| | SGR | | | | | | | |
| P0000528 | PGSALELRY | 0.092 | 15 | 4 | — | 53.3% | 43.8% | No toxic |
| | SQAPTVPSG | | | | | | | (non- |
| | AHLDPYVAG | | | | | | | toxic) |
| | SGR | | | | | | | |
| P0000527 | PGSALELRY | 0.092 | 16 | 4 | — | 53.3% | 43.8% | No toxic |
| | SQAPTVPSG | | | | | | | (non- |
| | AHLDPYVAG | | | | | | | toxic) |
| | SGR | | | | | | | |

A toxicity prediction result of a peptide P0001017 with a highest score is non-toxic (no toxic), and there are no drug peptide and active peptide component in the antigenic peptide, but homology of the antigenic peptide with the normal human protein sequence is over 80%, so it is determined that the antigenic peptide is not available, and a subsequent antigenic peptide is continued. A subsequent P0001016 is consistent with P0001017, and selection is continued downwards.

P0000578 meets requirements of toxicity prediction of non-toxicity, no drug peptide or active peptide component in the antigen peptide, and no more than 80% of homology with the normal human protein sequence, and P0000578 is selected and selecting is continued downwards.

Homology of P0000771 to P0000774 does not meet the requirements, which are all skipped.

P0000526 meets three requirements of toxicity prediction of non-toxicity, no drug peptide or active peptide component in the antigen peptide, and no more than 80% of homology with the normal human protein sequence, and P0000526 is selected and meanwhile P0000526 to P0000528 are all from a same variation, so P0000527 and P0000528 are excluded.

After above screening, following antigenic peptides are finally screened out from 10 antigenic peptides, as shown in a following table 3:

TABLE 3

| PID | VacSeq | iNeo_Score | VacRank | MutRank | Active | Drug | Hom | Toxic |
|---|---|---|---|---|---|---|---|---|
| P0000578 | KKLVFLLPAALFP | 0.293 | 3 | 2 | — | 27.6% | 51.7% | no toxic |
| | RKKLFITAAIVKK | | | | | | | |
| | KKK | | | | | | | |
| P0000526 | PGSALELRYSQAP | 0.092 | 14 | 4 | — | 53.3% | 43.8% | no toxic |
| | TVPSGAHLDPYVA | | | | | | | |
| | GSGR | | | | | | | |

In step 4, a next antigenic peptide is continued to be selected downward in the descending order, and above steps are repeated until enough candidate antigenic peptides are obtained or all of candidate antigenic peptides are selected so as to obtain screened antigenic peptides;
In step 5, a polypeptide is synthesized, a part with purity greater than threshold purity is collected in segments with a purification system, which is concentrated and freeze-dried after a sufficient amount is collected, freeze-dried products is placed for a certain period of time (about 7 to 14 days) at 4° C. for purity detection after drying, and if purity of this peptide is still higher than the threshold purity, the peptide is selected into a preparation group, and if the purity of the peptide is lower than the threshold purity, the peptide is determined as an unstable peptide and the peptide is taken into the preparation group.

As a preference, in synthesizing the polypeptide, solid-phase synthesis technology is adopted to produce high-purity peptides. A trifluoroacetic acid purification system is adopted as the purification system, with the threshold purity of 95. The part with purity greater than 95% is collected in segments, which is concentrated and freeze-dried after the sufficient amount is collected, the freeze-dried products is placed for 1 to 2 weeks at 4° C. for detection after drying. If the purity of this peptide is higher than 95%, it can be selected into the preparation group. If the purity of the peptide is lower than 95%, it may be determined as an unstable peptide and not selected into the preparation group. It should be noted that selection of a synthesis method and the purification system of the polypeptide is not limited, and the method adopted in the present disclosure is only an optimization, any case where the antigen peptide is screened by the method of the present disclosure is within protection scope of the present disclosure.

In step 6, grouping procedure design of the individualized tumor neoantigen polypeptide.

Specific rules for grouping the screened antigenic peptides into preparation groups in step 6 are as follows:
In a first step, the number of the antigen peptides is known to be p, and according to grouping rules, the number of the groups is determined to be g, and the number of the antigen peptides in each group is expressed by aᵢ, i being 1, 2, 3, . . . , g; a₁+a₂+ . . . +a_{g}=p. The computer system can list all possible grouping results (a₁, a₂, . . . , a_{g}) of polypeptides divided into each group according to two data of p and g, the system can calculate variance of each group according to grouping results of the polypeptides divided into each group, and rank all of the grouping results of the polypeptides according to the variance in a descending order. The more average the grouping results are, the smaller the variance is, and a group with a most average grouping result has a smallest variance and can be ranked first. Then, according to a variance ranking order, the system sequentially perform full permutation C_{P}^{a}^{₁} C_{P-a}_{₁}^{a}^{₂} C_{P-a}_{₁}₋ₐ_{₂}^{a}^{₃} . . . C_{P-a}_{₁}₋ₐ_{₂}₋ₐ_{₃} _{. . . -a}_{_{g-1}}^{a}^{_{g}} on the grouping results of the polypeptides divided into each group while checking conditions in second, third and fourth steps. When calculating the grouping results of the polypeptides divided into each group, the system quickly finds largest HPLC retention time of an antigen peptide that can be successfully grouped with dichotomy and groups the antigen peptide, and if the antigen peptide cannot be found, a next grouping result of polypeptides is calculated until grouping is successful;
in a second step, it is checked whether polypeptides with cysteine are evenly distributed in each group, and if the polypeptides with cysteine are not evenly distributed in each group, grouping is continued, if the polypeptides with cysteine are evenly distributed in each group;
in a third step, it is checked whether a gel chromatography polymer retention time corresponding to each peptide overlaps with retention time of a main peptide, and if the gel chromatography polymer retention time corresponding to each peptide does not overlap with the retention time of the main peptide, requirements are met;
if the retention time of the gel chromatography polymer corresponding to each peptide overlaps with retention time of the main peptide, grouping is continued until a successful grouping is obtained; and
in a fourth step, according to a formula of vaccine preparation, solubility of the preparation group in the third step is rechecked, and if the preparation group is soluble, the grouping is successful, and if the preparation group is insoluble, a grouping method in the third step is repeated for a second time until the solubility rechecking result indicates soluble so as to obtain successful grouping.

By way of example, with a primary batch containing 20 peptides, it is divided into 4 groups, and maximum retention time of two adjacent separable polypeptides in the preparation group is 2 min. It is assumed that data in group A of the primary batch includes A1, A2, A3 and A4; data in group B of the primary batch includes B1, B2, B3 and B4; data in group C of the primary batch includes C1, C2, C3 and C4; and data of group D of the primary batch includes D1, D2, D3 and D4.

As for a condition after successful grouping, in order to meet a condition of retention time difference, it must be met that A2-A1>2 min, A3-A2>2 min, A4-A3>2 min, B2-B1>2 min, B3-B2>2 min, B4-B3>2 min, C2-C1>2 min and C3-C2>2 min, C4-C3>2 min, D2-D1>2 min, D3-D2>2 min, D4-D3>2 min, the polypeptides with cysteine are evenly distributed to each group, groups A, B, C and D all contain 1-2 polypeptides with cysteine, retention time of all polymers for peptides divided into each group does not overlap with retention time of all of the peptides, polymer retention time of each of groups A, B, C and D does not overlap with a main peak of polypeptides, and solubility is rechecked according to the formula of the vaccine preparation. If it is soluble, the grouping is successful; and if it is insoluble, grouping is performed according to the above method for a second time until solubility rechecking result indicates soluble, so as to obtain successful grouping.

Objective: 20 peptides are designed for each person. Due to difference in a success rate of polypeptide synthesis and requirements of processes, an actual number of polypeptides used to prepare polypeptide preparations is different, with which the number of groups and the number of polypeptides in each group also vary and grouping rules also vary. In order to standardize grouping of polypeptide preparations, the grouping rules of the polypeptide preparations were formulated.

Grouping requirements for grouping procedure design include that:
1. n polypeptides are grouped and a number of groups is determined according to the number of the polypeptides, and specific rules are shown in Table 4 below.

TABLE 4

| Number of polypeptides | Number of groups |
|---|---|
| n > 20 | N being divided by 5 and then rounded up |
| 16 <= n <= 20 | 4 |
| 11 <= n <= 15 | 3 |
| 5 <= n <= 10 | 2 |

2. Because each peptide has a corresponding retention time, the peptides divided into each group are ranked according to corresponding HPLC retention time in an ascending order, and difference of retention time between two adjacent polypeptides in each group is greater than time difference corresponding to a maximum peak width of a single polypeptide peak.

For example, time difference corresponding to a maximum peak width of a single polypeptide peak of two adjacent polypeptides is greater than 2 min. It is assumed that the data in group A includes A1, A2, A3 and A4.

Then, in order to meet a condition of retaining time difference, it must be met that A2-A1>2 min, A3-A2>2 min, and A4-A3>2 min.
3. Polypeptides with cysteine are evenly divided into each group.

For example, five polypeptides with cysteine are divided into three groups, and distribution of these five peptides can only be 1-2-2, and it is not allowed to have more than two polypeptides in each group or have no polypeptide with cysteine in some group.
4. Each peptide has a corresponding gel chromatography polymer retention time, and it is required that gel chromatography polymer retention time corresponding to all of raw peptides divided into each group does not overlap with gel chromatography retention time of all of the raw peptides themselves.

Following embodiments are illustrated as an example.

In a specific implementation, permutation is adopted to obtain the results (for example, 15 peptides are divided into 4 groups, and full permutation CₘCₓC_{y}Cₙ is adopted, in which m+x+y+n=15). In order to obtain maximum retention time difference, difference of 5 minutes is firstly adopted, and maximum peak difference that can be successfully grouped can be quickly found with dichotomy. Then it is checked whether distribution requirement of the polypeptides with cysteine is met. After it is met, it is started to finally check whether the polymer retention time overlaps with the retention time of the main peptide; and if it is not met, it is continued to search for a next successful grouping, and a number of successful grouping can be selected so that when the number of successful grouping is reached, searching ends.

Manual rechecking can be performed as follows.

Solubility of a grouped preparation bottle group is checked according to corresponding raw material peptide combination and auxiliary materials. If it is soluble, grouping is successful, and if it is insoluble, grouping is performed for a second time according to above grouping steps until solubility rechecking result indicates soluble.

A vaccine preparation of the individualized tumor neoantigen peptide described above includes, in parts by mass, 1-3 parts of screened antigen peptide, 0-20 parts of inorganic salt and 10-100 parts of an excipient.

The excipient includes: a cosolvent, a filler or a tonicity adjusting agent. As an embodiment, the cosolvent includes saccharide such as mannitol, sorbitol, sucrose, trehalose, xylitol and dextran and polyol accessories; and as a preference, the cosolvent is mannitol. As an embodiment, the filler includes sucrose and lactose; and the tonicity adjusting agent is sodium chloride. It should be noted that meaning included in the present disclosure is a mixture of one or more of them.

As an embodiment, the vaccine preparation is a small-volume injection; a concentration of each antigenic peptide is 0.1-0.5 mg/ml, and a concentration of the cosolvent is 0.5%-5% (w/v). More preferably, the concentration of each antigenic peptide is 0.2-0.4 mg/ml, and the concentration of the cosolvent is 1%-3% (w/v).

As an embodiment, the vaccine preparation is freeze-dried powder injection; and a freeze-drying method includes following steps:
a, placing a bottle filled with polypeptide group liquid medicine into a freeze-drying dryer;
b, starting the freeze-drying dryer, and lowering a temperature of heat transfer oil of the freeze-drying dryer;
c, starting vacuumizing;
d, continuing to heat up a partition when vacuum reaches a specified requirement,
e, continuously adjusting the temperature of the heat transfer oil to heat up;
f, continuing to heat up the partition when vacuum reaches a specified requirement, and
g, closing the freeze-drying dryer, charging nitrogen, plugging and discharging.

Freeze-dried powder can be prepared with sterilized water for injection, 0.9% of sodium chloride solution or 5% of glucose solution, ringer's solution, lactated Ringer's solution and then administrated to patients.

### Embodiment 1: Preparation Process of Grouped Polypeptide Tumor Vaccine (Small-Volume Injection)

A solution containing 1% (W/V) of mannitol and 0.12 mmol of sodium chloride was prepared with water for injection, a group (5 pieces) of individualized antigenic peptides were weighed according to prescription, added, stirred and dissolved, and filtered aseptically with a 0.22 μm PVDF filter membrane, an appropriate amount of primary filtrate was discarded, and subsequent filtrate was collected. The subsequent filtrate was filled in medium borosilicate glass bottle at 1.0 ml/bottle, which was plugged, capped, visually inspected, labeled and packaged. The freeze-dried products were stored at −20° C.±5° C. Several other groups of individually designed antigenic peptides were prepared in a same way.

### Embodiment 2: Preparation Process of Grouped Polypeptide Tumor Vaccine (Freeze-Dried Powder Injection)

A solution containing 2% (W/V) of mannitol and 5% of sucrose (W/V) was prepared with water for injection, a group (4 pieces) of individualized antigenic peptides were weighed according to prescription, added, stirred and dissolved, and filtered aseptically with a 0.22 μm PVDF filter membrane, an appropriate amount of primary filtrate was discarded, and subsequent filtrate was collected. The subsequent filtrate was filled in medium borosilicate glass bottle at 1.0 ml/bottle, which was half-plugged, placed in a freeze-drying box that has been pre-cooled to about 10 degrees, and freeze-drying was started.

Freeze-drying: 1) pre-freezing: a shelf was cooled to −45° C. within 1 hour and maintained at −45° C. for 5 hours. 2) primary drying: vacuum was controlled below 0.2 mbar, a temperature was raised to −30° C. in 6 hours, and was maintained at −30° C. for 4 hours; and the vacuum was controlled to be below 0.1 mbar, the temperature was raised to −20° C. in 6 to 14 hours, and was maintained at −20° C. for 2 hours; and then the temperature was raised to 0° C. in 5 hours and was maintained at 0° C. for 1 hour. 3) secondary drying: the vacuum was controlled below 0.05 mbar, and the temperature is raised to 30° C. in 4 hours and was maintained at 30° C. for 6 hours. After freeze-drying, nitrogen was filled, plugging, capping, visually inspecting, labeling and packaging were performed. The freeze-dried products were stored at 5° C.±3° C.

Several other groups of individually designed antigenic peptides were prepared in a same way.

Experiment 1: Preliminary Stability of Polypeptide Tumor Vaccine (Powder Injection);

Ten types of raw peptides were divided into two preparation groups (each containing five peptides) according to grouping principle. Two batches of freeze-dried products were prepared as in Embodiment 2, and the prepared freeze-dried products were full and loose in appearance. The two batches of freeze-dried preparations were placed in a refrigerator at −20° C., 2 to 8° C. and a long-term stability investigation box at 25° C./RH60% for 30 days, respectively, and purity (in %) (with an area normalization method) of respective peptides, change of total impurities and solution condition after redissolution with sterilized water for injection were observed. Specifically, tables 5 and 6 below can be referred for details.

TABLE 5

| | | | | | | | Total | |
|---|---|---|---|---|---|---|---|---|
| Preparation | | | | | | | impurity | |
| group A | Polypeptide1 | Polypeptide 2 | Polypeptide3 | Polypeptide4 | Polypeptide 5 | Total | % | Redissolution |
| −20° C. | 6.76 | 28.91 | 18.52 | 19.64 | 22.76 | 96.6 | 3.4 | Clear |
| 2-8° C. 10 d | 6.82 | 28.85 | 18.60 | 19.73 | 22.76 | 96.8 | 3.2 | Clear |
| 2-8° C. 20 d | 6.87 | 28.94 | 18.73 | 19.56 | 22.92 | 97.0 | 3.0 | Clear |
| 2-8° C. 30 d | 6.63 | 28.58 | 18.37 | 19.52 | 22.98 | 96.1 | 3.9 | Clear |
| 25° C./RH60% | 6.74 | 28.51 | 18.38 | 19.71 | 22.71 | 96.1 | 3.9 | Clear |
| 10 d | | | | | | | | |
| 25° C./RH60% | 6.85 | 28.49 | 18.35 | 19.94 | 22.88 | 96.5 | 3.5 | Clear |
| 20 d | | | | | | | | |
| 25° C./RH60% | 6.83 | 28.51 | 18.51 | 19.68 | 22.83 | 96.4 | 3.6 | Clear |
| 30 d | | | | | | | | |

TABLE 6

| | | | | | | | Total | |
|---|---|---|---|---|---|---|---|---|
| Preparation | | | | | | | impurity | |
| group B | Polypeptide 1 | Polypeptide 2 | Polypeptide 3 | Polypeptide 4 | Polypeptide 5 | Total | % | Redissolution |
| −20° C. | 12.71 | 17.33 | 20.41 | 24.53 | 25.02 | 100.0 | 0.0 | Clear |
| 2 to 8° C. 10 d | 13.03 | 17.51 | 20.14 | 24.37 | 24.94 | 100.0 | 0.0 | Clear |
| 2 to 8° C. 20 d | 12.26 | 17.51 | 20.37 | 24.69 | 25.16 | 100.0 | 0.0 | Clear |
| 2 to 8° C. 30 d | 12.57 | 17.69 | 20.35 | 24.28 | 24.73 | 99.6 | 0.4 | Clear |
| 25° C./RH60% | 12.79 | 17.25 | 20.15 | 24.91 | 24.90 | 100.0 | 0.0 | Clear |
| 10 d | | | | | | | | |
| 25° C./RH60% | 12.88 | 17.21 | 20.10 | 24.34 | 25.46 | 100.0 | 0.0 | Clear |
| 20 d | | | | | | | | |
| 25° C./RH60% | 13.03 | 17.34 | 20.17 | 24.39 | 25.08 | 100.0 | 0.0 | Clear |
| 30 d | | | | | | | | |

Above preliminary stability results show that the total impurities in the two groups of polypeptide preparations do not increase significantly after being stored for 30 days under different storage conditions, and the solution remained clear after re-dissolution, indicating that the stability of the polypeptide freeze-dried preparations is good.

Experiment 2: Verification Experiment that the Screened Polypeptide Tumor Vaccine (Freeze-Dried Powder Injection) has Excellent Anti-Tumor Effect.
1. Validation of C57-B16F10 melanoma model
1) Establishment of murine tumor model C57-B16F10 melanoma model;

C57BL/6 mice, female, 6-8 weeks old, were purchased from SHANGHAI SLAC. B16-F10 murine melanoma tumor cells were inoculated. The tumor cells were counted before inoculation to ensure that cell viability is above 95%. Harvested B16-F10 murine melanoma tumor cells were injected subcutaneously into back of mice at a cell volume of 5×10⁴ cells/mouse.
2) Evaluation of tumor inhibition effect
a. tumor model grouping

Two days after tumor formation, 50 to 60 mice with similar tumor volumes and an average tumor diameter of about 0.3 cm were randomly divided into four groups, with at least 10 mice in each group, namely a negative control saline group, blank control adjuvant group, an adjuvant group and an iNeo-P01 group.
b. tumor model administration
Administration of the iNeo-P01 group:
before use, 4 bottles of iNeo-P01 preparation were dissolved with 300 ul of water for injection, with a content of 1 mg/ml.

When the C57BL/6 mice tumor grew to 50 mm³, basic immunization and enhanced immunization in two stages were performed. Polypeptide immunization was performed every three days for the first three times, and polypeptide immunization was performed every four days for the last three times, for a total of six times and with a dosage of polypeptide of 100 ug/time/mouse.

Tumor vaccine preparations were respectively injected subcutaneously into four parts of limbs of mice, with polypeptide being injected with mixed adjuvant GM-CSF each time, and an injection amount of GM-CSF is 2 μg/injection point, with a total of 4 injection points and a total of 8 ug. 100 μl of vaccine was inoculated at each site.

Administration of the negative control saline group, the blank control adjuvant group and the adjuvant group:

It was made in a basically same administration method as iNeo-P01. The saline group, blank control adjuvant group (1% of mannitol), and the adjuvant group (GM-CSF at 2 μg/injection point) were respectively injected subcutaneously into four parts of limbs of mice, with each part being inoculated with a volume of 100 μl.
c. sampling inspection and index evaluation

Two weeks after last administration, spleen cells, draining lymph node cells and tumor cells of mice were harvested to test various immune indexes of bodies.

Life cycle of all mice in the experimental group and the control group, as well as in different tumor model groups, were recorded and compared. Specific parameters to be compared include but are not limited to: overall survival (OS), tumor inhibition rate, CD4+IFN-λ+ cell ratio, CD8+IFN-λ+ cell ratio, etc.

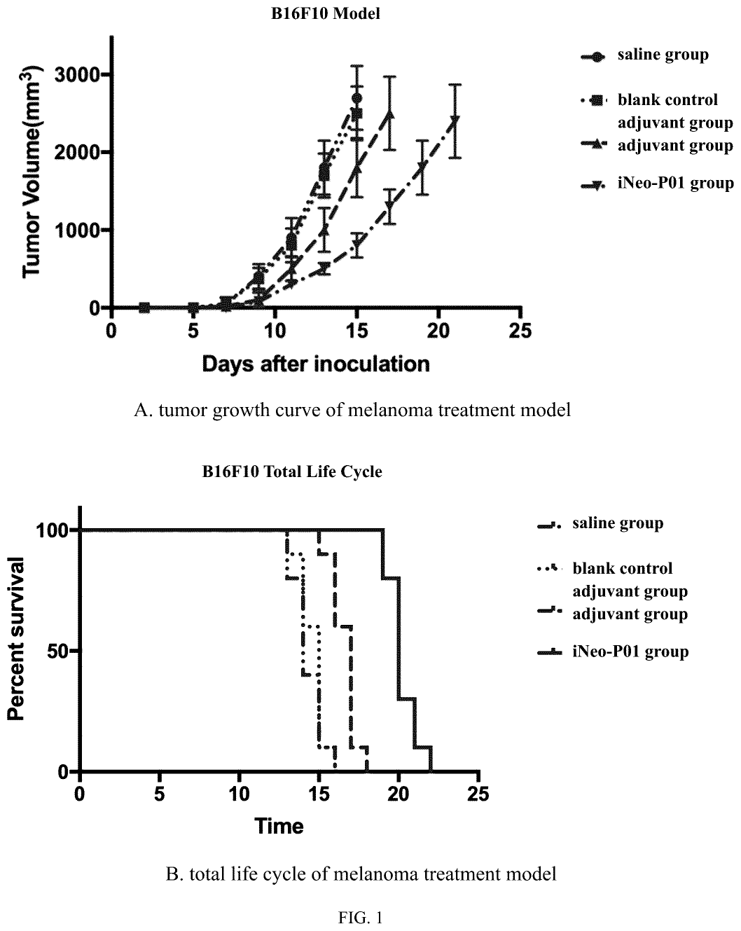

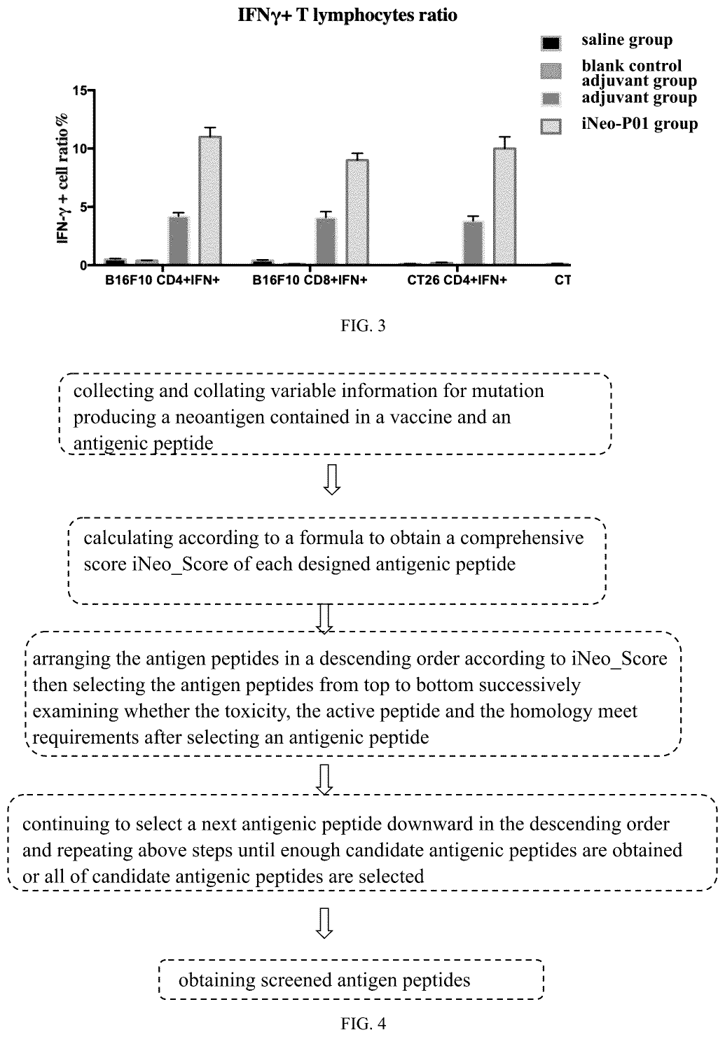

Results are shown in FIG. 1: treatment results of the C57-B16F10 melanoma model, and in FIG. 3: respective proportions of IFN-γ+ cells in the treatment model.

Results Analysis: By comparing results of the negative control saline group, the blank control adjuvant group, the adjuvant group and the iNeo-P01 group, it is proved that the iNeo-P01 group has good anti-tumor effect.
2. Validation of Balb/c-CT26 colon cancer model
1) Establishment of murine tumor model—BALB/C-CT26 colon cancer model;

Balb/c mice, female, 6-8 weeks old, were purchased from SHANGHAI SLAC. CT26 mouse-derived colon cancer cells were inoculated. The tumor cells were counted before inoculation to ensure that cell viability is above 95%. Harvested cells were injected subcutaneously into back of mice at a cell volume of 5×10⁴ cells/mouse.
2) Evaluation of tumor inhibition effect
a. tumor model grouping

Two days after tumor formation, 50 to 60 mice with similar tumor volumes and an average tumor diameter of about 0.3 cm were randomly divided into four groups, with at least 10 mice in each group, namely a negative control saline group, blank control adjuvant group, an adjuvant group and an iNeo-P01 group.
b. tumor model administration

Administration of the iNeo-P01 group
before use, 4 bottles of iNeo-P01 preparation were dissolved with 300 ul of water for injection, with a content of 1 mg/ml.

When the Balb/c mice tumor grew to 50 mm3, basic immunization and enhanced immunization in two stages were performed. Polypeptide immunization was performed every three days for the first three times, and polypeptide immunization was performed every four days for the last three times, for a total of six times and with a dosage of polypeptide of 100 ug/time/mouse.

Tumor vaccine preparations were respectively injected subcutaneously into four parts of limbs of mice, with polypeptide being injected with mixed adjuvant GM-CSF each time, and an injection amount of GM-CSF is 2 μg/injection point, with a total of 4 injection points and a total of 8 ug. 100 μl of vaccine was inoculated at each site.

Administration of the negative control saline group, the blank control adjuvant group and the adjuvant group:

It was made in a basically same administration method as iNeo-P01. The saline group, blank control adjuvant group (1% of mannitol), and the adjuvant group (GM-CSF at 2 μg/injection point) were injected respectively subcutaneously into four parts of limbs of mice, with each part being inoculated with a volume of 100 μl.
c. sampling inspection and index evaluation

One week after last administration, spleen cells, draining lymph node cells and tumor cells of mice were harvested to test various immune indexes of bodies.

Life cycle of all mice in the experimental group and the control group, as well as in different tumor model groups, were recorded and compared. Specific parameters to be compared include but are not limited to: overall survival (OS), tumor inhibition rate, CD4+IFN-λ+ cell ratio, CD8+IFN-λ+ cell ratio, etc.

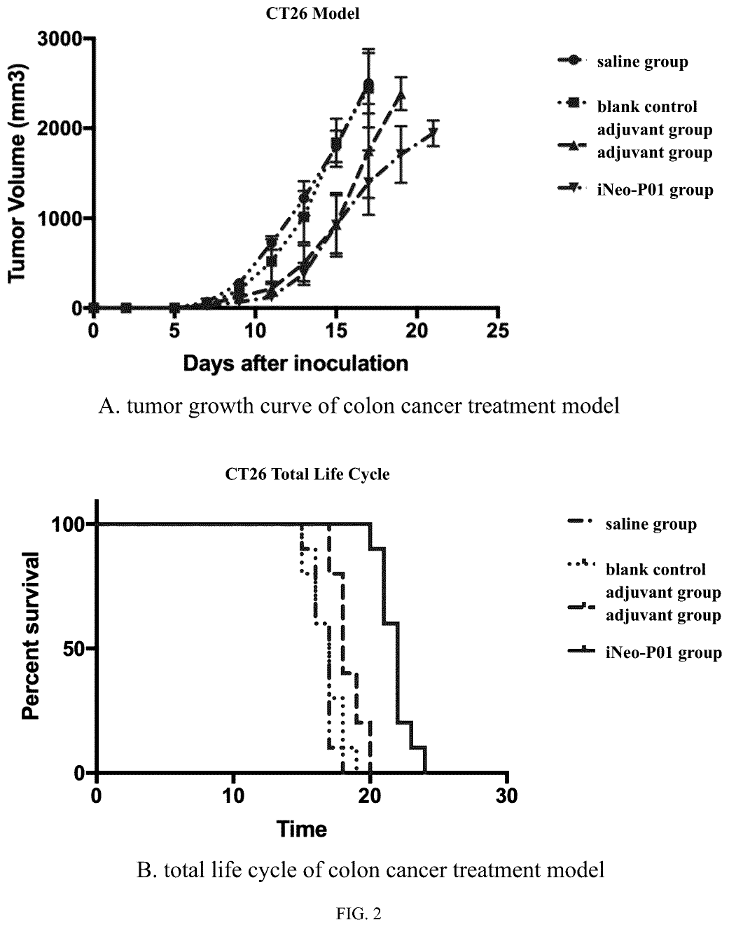

Results are shown in FIG. 2: treatment results of a Balb/c-CT26.wt colon cancer model in experiments of the disclosure (A: a tumor growth curve of the colon cancer treatment model; B: a total life cycle of the colon cancer treatment model), and in FIG. 3: respective proportions of IFN-γ+ cells in the treatment model.

Results Analysis: By comparing results of the negative control saline group, the blank control adjuvant group, the adjuvant group and the iNeo-P01 group, it is proved that the iNeo-P01 group has good anti-tumor effect.

Experiment 3: Solubilization of Mannitol on Individualized Tumor Neoantigen Peptides

Mannitol is a common adjuvant (skeleton forming agent) of freeze-dried preparation, which is used as a carrier to form a hard uniform skeleton so as to improve appearance of the freeze-dried preparation in glass bottles. Moreover, mannitol does not have Maillard reactions with residual amino group in the polypeptide, is an inert auxiliary material and can be used for subcutaneous injection. Hydroxyl groups in mannitol molecule can form hydrogen bonds with carbonyl groups in peptide bonds, which cannot directly promote dissolution of polypeptides, but can promote formation of micelle. Therefore, solubilization of mannitol is mainly solubility enhancement after micelle formation.

Results of solubility comparison test of the same polypeptide in water and mannitol solutions with different concentrations shown in table 7 as follows:

TABLE 7

| | | | Solubility | Solubility |
|---|---|---|---|---|
| | | | in 1% of | in 2% of |
| | | Solubility | mannitol | mannitol |
| | | in water | solution | solution |
| Number | Sequence | (mg/mL) | (mg/mL) | (mg/mL) |
| LXJI54 | TSLKGEIAFDPRRAYYLWF | <0.1 | ≥1 | ≥1 |
| WKYE17 | SVFGDRITGETIRSQNVMAAASK | <0.1 | ≥1 | ≥1 |
| WJIA11 | DIQDLKTCIASTTQNIEQKKK | <0.1 | ≥1 | ≥1 |
| GWMI10 | KKKGQQLSGVNAIYFYADQIYLS | <0.1 | ≥1 | ≥1 |
| | AGVPEEH | | | |
| YMZH19 | FITRPPHRFLSLLCLGLRIPQLSV | <0.1 | >1 | >1 |
| GWMI01 | KVVGADGVGKSALTIQLIQNH | <0.1 | ≥1 | ≥1 |
| GWMI13 | KLVFVRKSLNRIEFRECREEILKFL | <0.1 | ≥1 | ≥1 |
| | KK | | | |
| ZZJI44 | KKKKFVALVLSTLHMLTYGWTR | <0.1 | ≥1 | ≥1 |
| | AFEESRYK | | | |
| ZZJI58 | KEQMMREKEKLMLRLQDYEEKT | <0.1 | ≥1 | ≥1 |
| | KK | | | |
| LYLI37 | PGWSLTLGLRCSSRLGLPK | <0.1 | ≥1 | ≥1 |
| TZQI66 | KGFTQGDVGLAMGKLYRNDFSQ | <0.1 | ≥1 | ≥1 |
| | TTISRFEA | | | |
| TZQI51 | SSPAEFFELMKGQIRIAKRRVVMA | <0.1 | ≥1 | ≥1 |
| | SLYLGT | | | |
| YMZH10 | YKQLTIQVLLDRLVLQRLYPLAIQI | 0.3 | ≥1 | ≥1 |
| | KK | | | |
| HMZH59 | SRKLEDYGEQKSMSISTAKRLAEF | 0.3 | ≥1 | ≥1 |
| | LGDQK | | | |
| YMZH14 | KLTLASWGPKMVLLRLEHQFAVG | 0.3 | ≥1 | ≥1 |
| | K | | | |
| YMZH17 | FAEKLTLILQEKYKNYWYPEKPS | 0.3 | ≥1 | ≥1 |
| | KGQAY | | | |
| HMZH66 | KFKYVGTMEASEDATFNKITRSL | ≤1.5 | ≥10 | ≥10 |
| | QDLQQK | | | |
| HMZH51 | FHDQPHLKRKAVCSFQIYAVPWQ | <2 | ≥10 | ≥10 |
| | GT | | | |
| HMZH60 | GQAFKYSSNLLRHMRTHTGEKPF | <2 | ≥10 | ≥10 |

It can be seen that the solubility of tumor neoantigen peptides (with different amino acid sequences) obtained by individualized design in the mannitol solution is more than 3 times higher than that in water for injection. Especially for the peptides insoluble in water (with solubility lower than 0.1 mg/mL), with 1%-2% of mannitol solution for preparatiopm, their solubility can be can improved by more than 10 times, which fully meets solubility requirements of raw peptides in preparation production, and with simple and stable preparation technology, it is possible to apply vaccine preparations containing these tumor neoantigen peptides to clinic.

To sum up above experiments, it can be seen that the designed individualized tumor neoantigen peptide is screened and prepared into a preparation in the disclosure, which has excellent tumor inhibition effect; and in the disclosure, the mannitol can be used not only as a filler of freeze-dried preparations, but also as the cosolvent of insoluble polypeptides, thus greatly improving solubility of hydrophobic peptides in aqueous solvents.

The basic principles, main features and advantages of the present disclosure are shown and described in the above. It should be understood by those skilled in the industry that the above embodiments do not limit the present disclosure in any form, and all technical solutions obtained by equivalent substitution or equivalent transformation fall within the protection scope of the present disclosure.

1. A screening method of an individualized tumor neoantigen peptide, comprising following steps:
step 1, collecting and collating variable information for mutation producing a neoantigen contained in a vaccine and an antigenic peptide;
the variable information comprising: a mutation frequency Ag of the mutation producing the neoantigen at a genome level, a mutation frequency Ar of the mutation producing the neoantigen at a transcriptome level, expression level E of a gene where the mutation producing the neoantigen is located, a number H of amino acid changes caused by the mutation, quality indexes Mᵢ and Mᵢᵢ of epitope sequences of MHC protein type I and MHC protein type II, a situation ACTIVE where the antigen peptide contains active peptide, a situation DRUG where the antigen peptide contains drug peptide, homology HOM of the antigen peptide with normal human protein, and toxicity prediction TOXIC of the antigen peptide;
step 2, calculating according to a formula to obtain a comprehensive score iNeo_Score of each designed antigenic peptide;
the comprehensive score iNeo_Score being calculated as follows:
iNeo_Score=f₁(Ag)×f₂(Ar)×f₃(E)×f₄(Mᵢ)×f₅(H)+f₆(Mᵢᵢ);
where Ag represents the mutation frequency of the mutation producing the neoantigen at the genome level, Ar represents the mutation frequency of the mutation producing the neoantigen at the transcriptome level, E represents the expression level of the gene where the mutation producing the neoantigen is located, H represents the amino acid change caused by the mutation, Mᵢ and Mᵢᵢ represent type I and type II quality indexes of epitope sequences calculated by combining the epitope sequences of MHC protein type I and MHC protein type II, and f₁ to f₆ represent conversion functions of respective indexes;
removing an antigenic peptide with iNeo_Score of 0, and taking all of remaining antigenic peptides as candidate antigenic peptides for antigenic peptide screening;
step 3, arranging the antigen peptides in a descending order according to iNeo_Score, and then selecting the antigen peptides from top to bottom successively; and examining three indexes, namely, a toxicity, an active peptide and a homology according to peptide segment information after selecting an antigenic peptide;
directly discarding the antigenic peptide if any of above three indexes of the antigenic peptide is unqualified; and
reserving the antigen peptide as a selected antigen peptide and deleting other antigen peptide sequences generated by a same mutation if all of the above three indexes of the antigenic peptide are qualified;
step 4, continuing to select a next antigenic peptide downward in the descending order, and repeating above steps until enough candidate antigenic peptides are obtained or all of candidate antigenic peptides are selected so as to obtain screened antigenic peptides;
step 5, synthesizing a polypeptide, collecting a part with purity greater than threshold purity in segments with a purification system, which is concentrated and freeze-dried after a sufficient amount is collected, placing freeze-dried products for purity detection after drying, and if purity of this peptide is still higher than the threshold purity, selecting the peptide into a preparation group, and if the purity of the peptide is lower than the threshold purity, determining the peptide as an unstable peptide and taking the peptide into the preparation group; and
step 6, grouping the screened antigenic peptides into the preparation group;
grouping requirements comprising that:
1. n polypeptides are grouped and a number of groups is determined according to a number of the polypeptides;
2. peptides divided into each group are ranked according to corresponding HPLC retention time in an ascending order according to HPLC retention time corresponding to each peptide, and difference of retention time between two adjacent polypeptides in each group is greater than time difference corresponding to a maximum peak width of a single polypeptide peak;
3. polypeptides with cysteine are evenly divided into each group; and
4. each peptide has a corresponding gel chromatography polymer retention time, and it is required that gel chromatography polymer retention time corresponding to all of raw peptides divided into each group does not overlap with gel chromatography retention time of all of the raw peptides themselves.

2. The screening method of the individualized tumor neoantigen peptide according to claim 1,
wherein information sources of variables comprise:
information source of the mutation frequency Ag of the mutation producing the neoantigen at the genome level being exon sequencing,
information source of the mutation frequency Ar of the mutation producing the neoantigen at the transcriptome level being transcriptome sequencing,
information source of the expression level E of the gene where the mutation producing the neoantigen is located being transcriptome sequencing,
information source of a number H of amino acid changes caused by the mutation being mutation information annotation,
the quality indexes Mi and Mii of the epitope sequences of the MHC protein type I and MHC protein type II comprehensively considering a number of the epitope sequences, affinity of the epitope sequences with MHC protein, and affinity change of the epitope sequences with MHC protein; and
their information source being the affinity prediction of the epitope sequences with MHC protein, information source of the situation ACTIVE where the antigen peptide contains active peptide being antigenic peptide information annotation,
information source of the situation DRUG where the antigen peptide contains drug peptide being the antigenic peptide information annotation,
information source of homology HOM of the antigen peptide with normal human protein being the antigenic peptide information annotation, and
information source of toxicity prediction TOXIC of the antigen peptide being toxicity prediction analysis of the antigenic peptide.

3. The screening method of the individualized tumor neoantigen peptide according to claim 1,
wherein the three indexes, namely, a toxicity, an active peptide and a homology, in the step 3 are respectively as follows:
the active peptide index: amino acid sequences of the antigen peptide contains an active peptide amino acid sequence or a drug peptide amino acid sequence, and the active peptide amino acid sequence or the drug peptide amino acid sequence contains amino acid sites for mutation;
the toxicity index: the toxicity prediction of the antigen peptide is toxic; and
the homology index: homology of the antigenic peptide with human protein other than the gene where the mutation is located is over 80%.

4. The screening method of the individualized tumor neoantigen peptide according to claim 1,
wherein specific rules for n polypeptides being grouped and a number of groups being determined according to a number of the polypeptides in the step 6 are as follows:
a number of antigenic peptides is set to be n,
if the number of the antigenic peptides is n>20, the number of groups is a value of n divided by 5 and then rounded up;
if the number of the antigenic peptides is 16<=n<=20, the number of groups is 4;
if the number of the antigenic peptides is 11<=n<=15, the number of groups is 3; and
if the number of the antigenic peptides is 5<=n<=10, the number of groups is 2.

5. The screening method of the individualized tumor neoantigen peptide according to claim 1, wherein specific rules for grouping the screened antigenic peptides into the preparation group in step 6 are as follows:
in a first step,
the number of the antigen peptides is known to be p, and according to grouping rules, the number of the groups is determined to be g, and the number of the antigen peptides in each group is expressed by aᵢ, i being 1, 2, 3, . . . , g; a₁+a₂+ . . . +a_{g}=p. The computer system can list all possible grouping results (a₁, a₂, . . . , a_{g}) of polypeptides divided into each group according to two data of p and g, the system can calculate variance of each group according to grouping results of the polypeptides divided into each group, and rank all of the grouping results of the polypeptides according to the variance in a descending order. The more average the grouping results are, the smaller the variance is, and a group with a most average grouping result has a smallest variance and can be ranked first. Then, according to a variance ranking order, the system sequentially perform full permutation C_{P}^{a}^{₁} C_{P-a}_{₁}^{a}^{₂} C_{P-a}_{₁}₋ₐ_{₂}^{a}^{₃} . . . C_{P-a}_{₁}₋ₐ_{₂}₋ₐ_{₃} _{. . . -a}_{_{g-1}}^{a}^{_{g}} on the grouping results of the polypeptides divided into each group while checking conditions in second, third and fourth steps. When calculating the grouping results of the polypeptides divided into each group, the system quickly finds largest HPLC retention time of an antigen peptide that can be successfully grouped with dichotomy and groups the antigen peptide, and if the antigen peptide cannot be found, a next grouping result of polypeptides is calculated until grouping is successful;
in a second step, it is checked whether polypeptides with cysteine are evenly distributed in each group, and if the polypeptides with cysteine are not evenly distributed in each group, grouping is continued, if the polypeptides with cysteine are evenly distributed in each group, next step;
in a third step, it is checked whether the gel chromatography polymer retention time corresponding to each peptide overlaps with retention time of a main peptide, and if the gel chromatography polymer retention time corresponding to each peptide does not overlap with the retention time of the main peptide, requirements are met;
if the retention time of the gel chromatography polymer corresponding to each peptide overlaps with retention time of the main peptide, grouping is continued until a successful grouping is obtained; and
in a fourth step, according to a formula of vaccine preparation, solubility of the preparation group in the third step is rechecked, and if the preparation group is soluble, the grouping is successful, and if the preparation group is insoluble, a grouping method in the third step is repeated for a second time until the solubility rechecking result indicates soluble so as to obtain successful grouping.

6. A vaccine preparation of the individualized tumor neoantigen peptide according to claim 1, comprising, in parts by mass, 1-3 parts of screened antigen peptide, 0-20 parts of inorganic salt and 10-100 parts of an excipient.

7. The vaccine preparation of the individualized tumor neoantigen peptide according to claim 6, wherein the excipient comprises a cosolvent, a filler and a tonicity adjusting agent.

8. The vaccine preparation of the individualized tumor neoantigen peptide according to claim 7, wherein the cosolvent comprises: saccharide or polyol adjuvant.

9. The vaccine preparation of the individualized tumor neoantigen peptide according to claim 8, wherein the cosolvent is mannitol.

10. The vaccine preparation of the individualized tumor neoantigen peptide according to claim 7, wherein the vaccine preparation is a small-volume injection; a concentration of each antigenic peptide is 0.1-0.5 mg/ml, and a concentration of the cosolvent is 0.5%-5% (w/v).

11. The vaccine preparation of the individualized tumor neoantigen peptide according to claim 6, wherein the vaccine preparation is freeze-dried powder injection; and a freeze-drying method comprises following steps:
a, placing a bottle filled with vaccine liquid medicine into a freeze-drying dryer;
b, starting the freeze-drying dryer, and lowering a temperature of heat transfer oil of the freeze-drying dryer;
c, starting vacuumizing;
d, continuing to heat up a partition when vacuum reaches a specified requirement,
e, continuously adjusting the temperature of the heat transfer oil to heat up;
f, continuing to heat up the partition when vacuum reaches a specified requirement, and
g, closing the freeze-drying dryer, charging nitrogen, plugging and discharging.
